(19) United States
(12) Patent Application Publication
(10) Pub. No.: US 2012/0034146 A1
(43) Pub. Date: Feb. 9, 2012

(54) CARRIER FLUIDS FOR ABRASIVES

(75) Inventors: Hans-Peter SEELMANN-EGGEBERT, Limburgerhof (DE); Andreas SENF, Hettenleidelheim (DE); Markus Roesch, Oppenheim (DE); Joachim BENTELE, Ludwigshafen (DE); Kati SCHMIDT, Shanghai (CN); Udo STEFANOWSKI, Ludwigshafen (DE)

(73) Assignee: BASF SE, Ludwigshafen (DE)

(21) Appl. No.: 13/195,457

(22) Filed: Aug. 1, 2011

Related U.S. Application Data
(60) Provisional application No. 61/370,112, filed on Aug. 3, 2010.

(51) Int. Cl. C01B 33/02; C09K 3/14; H01L 21/78; C07D 303/04

(52) U.S. Cl. 423/348; 549/512; 51/309; 51/307; 451/36; 438/460; 257/E21.599

(57) ABSTRACT

The invention relates to improved novel carrier fluids for abrasives, in particular cutting fluids, for wafer production, the use thereof and a method of cutting wafers.

The invention relates to the use of modified polyglycols for producing carrier fluids for abrasives, novel carrier fluids for abrasives, in particular cutting fluids, the use of the carrier fluid in the removal of material, in particular in the cutting of wafers and also wafers produced with the aid of the cutting fluid.

Abrasives, also known as grinding agents or abrasive materials, are materials, preferably grains of hard material, which are utilized for removing a material. The use of abrasives as a dispersion in fluids, for example grinding fluids or cutting fluids, is known. Abrasives can in this way be used for polishing wafers, for example silicon wafers, and also for polishing plastics, for example for lenses. Furthermore, the use of abrasives in cutting fluids for cutting wafers is also known. Wafers are thin slices of semiconductors which are used, for example, in photovoltaics. Electronic components, especially integrated circuits, can be produced from wafers. The wafers generally comprise a brittle material, for example silicon, but may also be made of gallium arsenide or cadmium telluride, etc. Wafers are generally produced from cylindrical or cubic monocrystals or polycrystals which are sawn into the individual slices, viz. the wafers. The sawing (also referred as cutting or lapping) is an industrial practice carried out by wire sawing. This is a parting process using a thin wire as cutter and using unbounded cutting grains in a carrier fluid. The wire generally has a diameter of from 80 to 180 μm. It dips into a slurry of carrier fluid and cutting grain and draws the cutting grains adhering to the wire surface into the saw cut. The object/silicon block, known as ingot, to be sawn/lapped is cut by means of the cutting grains into wafers, with particles being removed from the solid to be cut. The carrier fluid for the cutting grains is applied together with the cutting grains as slurry via an immersion bath through which the wire runs or, in general, via nozzles. The carrier fluid has, inter alia, the task of effecting adhesion of the cutting grains to the wire and carrying particles of removed material from the solid to be parted. Furthermore, the carrier fluid has the task of providing cooling and transport of the abraded material through the saw cut.

A process for parting a workpiece, for example a wafer, by means of wire sawing is known from EP 1 757 419 A1; here, a slurry applied to the wire is used and the water content of at least part of the gaseous medium surrounding the slurry is regulated or controlled. Furthermore, the use of glycols as carrier substance is known from EP 1 757 419 A1.

A cutting oil comprising a) a polyether compound and b) silica particles and the use of this cutting oil composition for cutting an ingot using a wire saw, in particular for cutting silicon ingots, is known from DE 199 83 092 B4 and U.S. Pat. No. 6,383,991 B1.

Water-based lubricants based on polyethers are known from EP 0 131 657 A1 and U.S. Pat. No. 4,828,735. Cutting fluids are likewise known from the Chinese patent application CN 101205498 A; a reduction in the water uptake is not indicated here. The compounds specifically mentioned are polyalkyleneoxy compounds which are etherified with alcohols having from 1 to 4 carbon atoms.

EP 686 684 A1 discloses a sawing suspension comprising an abrasive material in an aqueous phase comprising one or more water-soluble polymers as thickeners. US 2007/0010406 A1 discloses hydroxy polyethers as additives for aqueous cutting fluids which can be used, inter alia, for producing silicon wafers.

The known cutting fluids are generally based on an aqueous or water-soluble base. However, the presence of water is disadvantageous because it can cause corrosion and it is also possible, for example, for hydrogen to be evolved when cutting silicon wafers as a result of reaction of water and silicon. An additional problem here is that silicate or polysilicate formation occurs on the wafer and in the slurry.

The known water-soluble systems, too, can comprise water and, owing to their microscopic properties, attract water so that the same disadvantages as in aqueous systems can occur.

It was an object of the invention to provide improved carrier fluids for abrasives, in particular cutting fluids, which, in particular, lead to a reduction in the water uptake and a reduction in the energy required for sawing.

The invention provides for the use of compounds of the formula I

R¹[O(EO)ₓ(AO)_{y}H]_{z}
where
R¹ is a z-valent alkyl radical having from 1 to 20 carbon atoms
(EO) is an ethyleneoxy radical
(AO) is an alkyleneoxy radical having from 3 to 10 carbon atoms
x is an integer from 3 to 12, in particular from 5 to 10
y is an integer from 0 to 10, in particular from 4 to 8
z is an integer from 1 to 6, in particular from 1 to 3,
   for producing carrier fluids for abrasives, in particular cutting fluids, having a reduced water uptake for removal of material, in particular for sawing wafers by means of a wire saw.

The invention further provides carrier fluids for abrasives, in particular cutting fluids, comprising at least one compound of the formula I

R¹[O(EO)ₓ(AO)_{y}H]_{z}
where
R¹ is a z-valent alkyl radical having from 5 to 10 carbon atoms
(EO) is an ethyleneoxy radical
(AO) is an alkyleneoxy radical having from 3 to 10 carbon atoms
x is an integer from 3 to 12, in particular from 5 to 10
y is an integer from 0.5 to 10, in particular from 4 to 8
z is an integer from 1 to 6, in particular from 1 to 3.

The invention further provides novel compounds II of the formula

R¹O(EO)ₓ(AO)_{y}H

where
R¹ is 2-methylbutyl or 3-methylbutyl
(EO) is an ethyleneoxy radical
(AO) is an alkyleneoxy radical having from 3 to 10 carbon atoms
x is an integer from 3 to 12, in particular from 5 to 10
y is an integer from 0 to 10, in particular from 4 to 8
z is an integer from 1 to 6, in particular from 1 to 3

In preferred compounds of the formula II, at least as many EO units as PO units are present. Very particularly preferred compounds of the formula II are shown in the following table:

| Compound | R¹ | x | y |
|---|---|---|---|
| II.1 | 2-methylbutyl | 5.5 | 5.5 |
| II.2 | 2-methylbutyl | 5.0 | 6.0 |

In a preferred embodiment, the ratio of x to y in the compound of the formulae I and II is equal to or less than 1.

For the purposes of the present invention, compounds of the formula II are particularly preferred embodiments of compounds of the formula I.

In a preferred embodiment,
in formula I: R¹ is a pentyl radical, preferably
   H₃C—CHCH₃—CH₂—CH₂— (3-methylbutyl) and
   H₃C—CH₂—CHCH₃—CH₂— (2-methylbutyl), in particular at least 10% of 3-methylbutyl
In formulae I and II: AO is propyleneoxy, butyleneoxy and pentyleneoxy or a mixture thereof.

In the case of the compounds of the formulae I and II, the recurring units (EO) and (AO) can be present as a block or randomly distributed. In a preferred embodiment, they are randomly distributed. It has surprisingly been found that the viscosity of the compounds is largely independent of temperature when the recurring units (AO) and (EO) are randomly distributed. In particular, when the recurring units (EO) and (AO) are randomly distributed, the compounds of the formula I to be used according to the invention have a viscosity index in a slurry comprising 40% by weight of silicon carbide of the type Carborex F 800 PV from Washington Mills AS, NO-7300 Orkanger, Norway, of not more than 45%, preferably less than 30%, in particular less than 20%, with the viscosity index being defined as follows: the viscosity index is, for the purposes of the present invention, the percentage decrease in the viscosity of the compounds of the formula I at 50° C. compared to the viscosity at 30° C. The viscosity here is the dynamic viscosity (Brookfield, spindle V-73) determined in accordance with DIN EN 12092.

The carrier fluids of the invention can not only comprise one compound of the formula I but also mixtures of compounds of the formula I.

The preparation of compounds of the formula I is known per se, see, for example, Nonionic Surfactants, edited by Martin J. Schick, Volume 2, Chapter 4 (Marcel Dekker, Inc., New York 1967). The preparation of the novel compounds of the formula II can be carried out in an analogous way.

In a preferred embodiment, the carrier fluids for abrasives, in particular cutting fluids, consist of the compound of the formula I. The molecular weight of the compound of the formula I is preferably from 200 to 1200 g/mol. In a further preferred embodiment, alkylene alcohols based on ethylene oxides, propylene oxides or copolymers of ethylene oxides and propylene oxides, preferably having a molecular weight of from 200 to 800 g/mol are comprised in addition to the compounds of the formula I. In use for sawing, the cutting fluid comprises abrasives, in particular cutting grains, in addition to the compound of the formula I.

In a further preferred embodiment, the carrier fluids for abrasives, in particular cutting fluids, are combined with at least one further additive, in particular with at least one

monoalkylene, oligoalkylene or polyalkylene glycol,
wetting agent,
thickener,
dispersant,
corrosion inhibitor,
complexing agent and/or
other additives such as scale inhibitors
to form a carrier fluid.

Preference is given to at least one of the following additives being added in the following parts by weight per 100 parts by weight of the compound I:

alkylene glycols: from 10 to 90, in particular from 20 to 60, parts by weight
wetting agent: from 1 to 100, in particular from 10 to 40, parts by weight
thickener: from 0.5 to 20, in particular from 1 to 10, parts by weight
dispersant: from 0.1 to 20, in particular from 0.5 to 10, parts by weight
corrosion inhibitor: from 0.1 to 10, in particular from 0.1 to 3, parts by weight
complexing agent: from 0.1 to 10, in particular from 1 to 5, parts by weight
other additives: from 0.05 to 10, in particular from 0.1 to 5, parts by weight

The water content of the composition according to the invention is, based on the total composition, not more than 10% by weight, preferably not more than 5% by weight, in particular less than 1% by weight.

Particularly preferred additives are indicated below:

### Wetting Agents

In addition to the compounds of the formula I to be used according to the invention, it is possible to use further wetting agents, in particular

### (1) Poly(Oxyalkylene) Derivatives of

a) sorbitan esters, e.g. poly(oxyethylene)sorbitan monolaurate, poly(oxyethylene)sorbitan monooleate, poly(oxyethylene)sorbitan trioleate
b) fatty amines, e.g. tallow amino ethoxylates, soy amino ethoxylates,
c) castor oil, e.g. castor oil ethoxylates,
d) alkanolamides, e.g. coconut oil alkanolamide ethoxylates,
e) fatty acids, e.g. oleic acid ethoxylates, lauric acid ethoxylates, palmitic acid ethoxylates,
f) fatty alcohols,
g) linear alcohol ethoxylates, nonylphenol ethoxylates, octylphenol ethoxylates

### (2) Hydrophilic Polydimethylsiloxanes

a) poly(dimethyl)siloxane substituted by at least one carbonyl end group, poly(dimethyl)siloxane copolymers,
c) poly(dimethylsiloxane)-b-poly(propylene oxide)-b-poly(ethylene oxide) copolymers,
d) polyquarternary (dimethylsiloxane) copolymers

### (3) Fatty Imidazolines

### (4) Fatty Acid Esters of

a) phosphates,
b) sorbitans,
c) glycerol compounds, e.g. glyceryl monooleate, glyceryl dioleate, glyceryl trioleate, dilaurate,
e) sulfosuccinic acid,

### (5) Quaternary Compounds e.g.

quaternary ammonium methosulfate.

Further suitable nonionic, cationic, anionic or amphoteric wetting agents are, in particular
alkoxylated C₄-C₂₂-alcohols such as fatty alcohol alkoxylates or oxo alcohol alkoxylates. These can be alkoxylated by ethylene oxide, propylene oxide and/or butylene oxide. All alkoxylated alcohols which have at least two molecules of one of the abovementioned alkylene oxides added on can be used as wetting agents. Possible compounds of this type are block polymers of ethylene oxide, propylene oxide and/or butylene oxide or addition products which comprise the abovementioned alkylene oxides distributed randomly or in blocks. The nonionic wetting agents generally comprise from 2 to 50 mol, preferably from 3 to 20 mol, of at least one alkylene oxide per mole of alcohol. The alcohols preferably have from 10 to 18 carbon atoms. Depending on the type of alkoxylation catalyst used in the preparation, the method of preparation and the work-up, the alkoxylates have a broad or narrow alkylene oxide homologue distribution;
alkylphenol alkoxylates such as alkylphenol ethoxylates having C₆-C₁₄-alkyl chains and from 5 to 30 alkylene oxide units;
alkyl polyglucosides having from 8 to 22, preferably from 10 to 18, carbon atoms in the alkyl chain and generally from 1 to 20, preferably from 1.1 to 5, glucoside units, sorbitan alkanoates, also alkoxylated;
N-alkylglucamides, fatty acid alkoxylates, fatty acid amine alkoxylates, fatty acid amide alkoxylates, fatty acid alkanolamide alkoxylates, alkoxylated, block copolymers of ethylene oxide, propylene oxide and/or butylene oxide, polyisobutene ethoxylates, polyisobutene-maleic anhydride derivatives, optionally alkoxylated monoglycerides, glyceryl monostearates, sorbitan esters and bisglycerides.

Particularly useful nonionic wetting agents are alkyl alkoxylates or mixtures of alkyl alkoxylates, as are described, for example, in DE-A 102 43 363, DE-A 102 43 361, DE-A 102 43 360, DE-A 102 43 365, DE-A 102 43 366, DE-A 102 43 362 or DE-A 43 25 237. These are alkoxylation products obtained by reaction of alkanols with alkylene oxides in the presence of alkoxylation catalysts or mixtures of alkoxylation products. Particularly suitable starter alcohols are the Guerbet alcohols, in particular ethylhexanol, propylheptanol and butyloctanol. Particular preference is given to propylheptanol. Preferred alkylene oxides are propylene oxide and ethylene oxide, with alkyl alkoxylates having a direct bond between a preferably short polypropylene oxide block and the starter alcohol, as are described, for example, in DE-A 102 43 365, being particularly preferred because of their low residual alcohol content and their good biodegradability.

A preferred class of suitable nonionic wetting agents are the alcohol alkoxylates of the general formula (NI)

R¹—O—(CH₂—CHR⁵—O—)ᵣ(CH₂—CH₂—O—)ₙ(CH₂—CHR⁶—O—)ₛ(CH₂—CHR²—O—)ₘH (NI)

where
R¹ is an at least singly branched C₄₋₂₂-alkyl or -alkylphenol,
R² is C₃₋₄-alkyl,
R⁵ is C₁₋₄-alkyl,
R⁶ is methyl or ethyl,
n is an average value of from 1 to 50,
m is an average value of from 0 to 20, preferably from 0.5 to 20,
r is an average value of from 0 to 50,
s is an average value of from 0 to 50,
where n is at least 0.5 when R⁵ is methyl or ethyl or r is 0.

A mixture of from 20 to 95% by weight, preferably from 30 to 95% by weight, of at least one abovementioned alkyl alkoxylate and from 5 to 80% by weight, preferably from 5 to 70% by weight, of a corresponding alcohol alkoxylate in which R¹ is, however, an unbranched alkyl radical having the same number of carbon atoms is also possible.

Furthermore, alcohol alkoxylates of the general formula (NII)

R³—O—(CH₂—CH₂—O)ₚ(CH₂—CHR⁴—O—)_{q}H (NII)

where
R³ is branched or unbranched C₄₋₂₂-alkyl or -alkylphenol,
R⁴ is C₃₋₄-alkyl,
p is an average value of from 1 to 50, preferably from 4 to 15,
q is an average value of from 0.5 to 20, preferably from 0.5 to 4, more preferably from 0.5 to 2,
are also suitable.

A mixture of from 5 to 95% by weight of at least one branched alcohol alkoxylate (NII) as described directly above and from 5 to 95% by weight of a corresponding alcohol alkoxylate in which, however, an unbranched alkyl radical is present in place of a branched alkyl radical is also possible.

In the alcohol alkoxylates of the general formula (NI), R² is preferably propyl, in particular n-propyl.

In the alcohol alkoxylates of the general formula (NII), n preferably has an average value of from 4 to 15, particularly preferably from 6 to 12, in particular from 7 to 10.

m preferably has an average value from 0.5 to 4, particularly preferably from 0.5 to 2, in particular from 1 to 2.

The radical R¹ is preferably a C₈₋₁₅-, particularly preferably C₈₋₁₃-, in particular C₈₋₁₂-alkyl radical which is at least singly branched. It is also possible for a plurality of branches to be present.

R⁵ is preferably methyl or ethyl, in particular methyl.
R⁶ is preferably ethyl.

In the mixtures, compounds having unbranched and branched alcohol radicals R¹ are present. This is the case, for example, for oxo alcohols which have a proportion of linear alcohol chains and a proportion of branched alcohol chains. For example, a C_{13/15} oxo alcohol frequently has about 60% by weight of completely linear alcohol chains together with about 40% by weight of α-methyl-branched and C_{≧2}-branched alcohol chains.

In the alcohol alkoxylates of the general formula (NII), R³ is preferably a branched or unbranched C₈₋₁₅-alkyl radical, particularly preferably a branched or unbranched C₈₋₁₃-alkyl radical and in particular a branched or unbranched C₈₋₁₂-alkyl radical. R⁴ is preferably propyl, in particular n-propyl. p preferably has an average value of from 4 to 15, particularly preferably an average value of from 6 to 12 and in particular an average value of from 7 to 10. q preferably has an average value of from 0.5 to 4, particularly preferably from 0.5 to 2, in particular from 1 to 2.

In a manner analogous to the alcohol alkoxylates of the general formula (NI), the alcohol alkoxylates of the general formula (NII) can also be present as mixtures having unbranched and branched alcohol radicals.

Possible alcohol components on which the alcohol alkoxylates are based include not only pure alkanols but also homologous mixtures having a range of carbon atoms. Examples are C_{8/10}-alkanols, C_{10/12}-alkanols, C_{13/15}-alkanols, C_{12/15}-alkanols. Mixtures of a plurality of alkanols are also possible.

The above alkanol alkoxylates or mixtures according to the invention are preferably prepared by reacting alcohols of the general formula R¹—OH or R³—OH or mixtures of corresponding branched and unbranched alcohols optionally firstly with C₃₋₆-alkylene oxide, then with ethylene oxide and subsequently optionally with C₃₋₄-alkylene oxide and then with an appropriate C₅₋₆-alkylene oxide. The alkoxylations are preferably carried out in the presence of alkoxylation catalysts. In particular, basic catalysts such as potassium hydroxide are used here. The random distribution of the amounts of the alkylene oxides incorporated can be greatly restricted by means of specific alkoxylation catalysts such as modified bentonites or hydrotalcites as are described, for example, in WO 95/04024, so that “narrow-range” alkoxylates are obtained.

In a particular embodiment of the present invention, the alkoxylates are alkoxylate mixtures comprising alkoxylates of the general formula (NII)

C₅H₁₁CH(C₃H₇)CH₂O(B)ₚ(A)ₙ(B)ₘ(A)_{q}H (NIII)

where
A is ethyleneoxy,
the radicals B are each, independently of one another, C₃₋₁₀-alkyleneoxy, preferably propyleneoxy, butyleneoxy, pentyleneoxy or mixtures thereof,
where groups A and B are present in the form of blocks in the order indicated,
p is from 0 to 10,
n is from >0 to 20,
m is from >0 to 20,
q is from >0 to 10,
p+n+m+q is at least 1,
where
from 70 to 99% by weight of alkoxylates A1 in which C₅H₁₁ is n-C₅H₁₁ and
from 1 to 30% by weight of alkoxylates A2 in which C₅H₁₁ is C₂H₅CH(CH₃)CH₂ and/or CH₃CH(CH₃)CH₂CH₂,
are present in the mixture.

In the general formula (NIII), p is from 0 to 10, preferably from 0 to 5, in particular from 0 to 3. If blocks (B)ₚ are present, p is preferably from 0.1 to 10, particularly preferably from 0.5 to 5, in particular from 1 to 3.

In the general formula (NIII), n is preferably in the range from 0.25 to 10, in particular from 0.5 to 7, and m is preferably in the range from 2 to 10, in particular from 3 to 6. B is preferably propyleneoxy and/or butyleneoxy, especially propyleneoxy in both positions.

q is preferably in the range from 1 to 5, particularly preferably in the range from 2 to 3.

The sum p+n+m+q is at least 1, preferably from 3 to 25, particularly preferably from 5 to 15, in particular from 7 to 13.

Preference is given to 3 or 4 alkylene oxide blocks being present in the alkoxylates. In one embodiment, firstly ethyleneoxy units, then propylene oxide units and then ethyleneoxy units are adjoined to the alcohol radical. In a further embodiment, firstly propyleneoxy units, then ethyleneoxy units, then propyleneoxy units and finally ethyleneoxy units are adjoined to the alcohol radical. It is also possible for the other alkyleneoxy units indicated to be present in place of the propyleneoxy units.

p, n, m and q are each a value averaged over the alkoxylates. For this reason, p, n, m, q can also have nonintegral values. The alkoxylation of alkanols generally gives a distribution of the degree of alkoxylation which can to a certain extent be set by use of different alkoxylation catalysts. The choice of appropriate amounts of the groups A and B enables the property spectrum of the alkoxylate mixtures according to the invention to be matched to practical requirements.

The alkoxylate mixtures are obtained by alkoxylation of the parent alcohols C₅H₁₁CH(C₃H₇)CH₂OH. The starting alcohols can be mixed from the individual components so to as give the ratio according to the invention. They can be prepared by aldol condensation of valeraldehyde and subsequent hydrogenation. The preparation of valeraldehyde and the corresponding isomers is carried out by hydroformylation of butene, as described, for example, in U.S. Pat. No. 4,287,370; Beilstein E IV 1, 32 68, Ullmanns Encyclopedia of Industrial Chemistry, 5th Edition, Volume A1, pages 323 and 328 ff. The subsequent aldol condensation is described, for example, in U.S. Pat. No. 5,434,313 and Römpp, Chemie Lexikon, 9th Edition, keyword “Aldol-Addition”, page 91. The hydrogenation of the aldol condensation product follows general hydrogenation conditions.

Furthermore, 2-propylheptanol can be prepared by condensation of 1-pentanol (as mixture of the corresponding 1-methylbutanols) in the presence of KOH at elevated temperatures, see, for example, Marcel Guerbet, C.R. Acad Sci Paris 128, 511, 1002 (1899). Reference may also be made to Römpp, Chemie Lexikon, 9th Edition, Georg Thieme Verlag Stuttgart, and the references cited therein and also Tetrahedron, Vol. 23, pages 1723 to 1733.

In the general formula (NIII), the radical C₅H₁₁ can be n-C₅H₁₁, C₂H₅CH(CH₃)CH₂ or CH₃CH(CH₃)CH₂CH₂. The alkoxylates are mixtures in which
from 70 to 99% by weight, preferably from 85 to 96% by weight, of alkoxylates A1 in which C₅H₁₁ is n-C₅H₁₁ are present and
from 1 to 30% by weight, preferably from 4 to 15% by weight, of alkoxylates A2 in which C₅H₁₁ is C₂H₅CH(CH₃)CH₂ and/or CH₃CH(CH₃)CH₂CH₂ are present.

The radical C₃H₇ is preferably n-C₃H₇.

The alkoxylates can also be block isotridecanol alkoxylates of the general formula (NV)

R—O—(CₘH₂ₘO)ₓ—(CₙH₂ₙO)_{y}—H (NV)

where
R is an isotridecyl radical,
m is 2 and at the same time n is 3 or 4 or
m is 3 or 4 and at the same time n is 2 and
x and y are, independently of one another, from 1 to 20,
where in the case of m=2/n=3 or 4, the variable x is greater than or equal to y.
These block isotridecanol alkoxylates are described, for example, in DE 196 21 843 A1.

Another suitable class of nonionic surfactants are end-capped alcohol alkoxylates, in particular of alcohol alkoxylates mentioned above. In a particular embodiment, the end-capped alcohol alkoxylates are the end-capped alcohol alkoxylates corresponding to the alcohol alkoxylates of the general formulae (NI), (NII), (NIII) and (NV). The end cap can be produced, for example, by means of dialkyl sulfate, C₁₋₁₀-alkyl halides, C₁₋₁₀-phenyl halides, preferably chlorides, bromides, particularly preferably cyclohexyl chloride, cyclohexyl bromide, phenyl chloride or phenyl bromide.

Examples of end-capped alkoxylates are also described in DE-A 37 26 121, the entire relevant disclosure of which is incorporated by reference into the present invention. In a preferred embodiment, these alcohol alkoxylates have the general structure (NVI),

R^{I}—O—(CH₂—CHR^{II}—O)_{m′}(CH₂—CHR^{III}O)_{n′}R^{IV} (NVI)
where
R^{I} is hydrogen or C₁-C₂₀-alkyl,
R^{II} and R^{III} are identical or different and are each, independently of one another, hydrogen, methyl or ethyl,
R^{IV} is C₁-C₁₀-alkyl, preferably C₁-C₄-alkyl, or cyclohexyl or phenyl,
m′ and n′ are identical or different and are each greater than or equal to 0,
with the proviso that the sum of m′ and n′ is from 3 to 300.

Another class of nonionic wetting agents are alkyl polyglucosides which preferably have from 6 to 22, particularly preferably from 10 to 18, carbon atoms in the alkyl chain. These compounds generally comprise from 1 to 20, preferably from 1.1 to 5, glucoside units.

Further possible nonionic wetting agents are the end-capped fatty acid amide alkoxylates of the general formula

R^{I}—CO—NH—(CH₂)_{y}—O-(A¹O)ₓ—R²

known from WO-A 95/11225, where
R^{I} is a C₅-C₂₁-alkyl or alkenyl radical,
R² is a C₁-C₄-alkyl group,
A^{I} is C₂-C₄-alkylene,
y is 2 or 3 and
x is from 1 to 6.

Examples of such compounds are the reaction products of n-butyl triglycolamine of the formula H₂N—(CH₂—CH₂—O)₃—C₄H₉ with methyl dodecanoate or the reaction products of ethyl tetraglycolamine of the formula H₂N—(CH₂—CH₂—O)₄—C₂H₅ with a commercial mixture of saturated C₈-C₁₈ methyl fatty acid esters.

Further suitable nonionic wetting agents are polyhydroxy or polyalkoxy fatty acid derivatives such as polyhydroxy fatty acid amides, N-alkoxy or N-aryloxy polyhydroxy fatty acid amides, fatty acid amide ethoxylates, in particular end-capped fatty acid amide ethoxylates, and also fatty acid alkanolamide alkoxylates.

Further suitable nonionic wetting agents are block copolymers of ethylene oxide, propylene oxide and/or butylene oxide (Pluronic® and Tetronic® grades from BASF SE and BASF Corp.). In a preferred embodiment, these copolymers are triblock copolymers having polyethylene/polypropylene/polyethylene blocks and a molecular weight of from 4000 to 16 000, with the proportion by weight of the polyethylene blocks being from 55 to 90%, based on the triblock copolymer. Particular preference is given to triblock copolymers having a molecular weight of more than 8000 and a polyethylene content of from 60 to 85% by weight, based on the triblock copolymer. These preferred triblock copolymers are, in particular, commercially available under the trade names Pluronic F127, Pluronic F108 and Pluronic F98, in each case from BASF Corp., and are described in WO 01/47472 A2, the entire relevant disclosure of which is incorporated by reference into the present invention.

In addition, preference is also given to using block copolymers of ethylene oxide, propylene oxide and/or butylene oxide capped at one or both ends. Capping at one end is achieved, for example, by using an alcohol, in particular a C₁₋₂₂-alkyl alcohol, for example methanol, as starting compound for the reaction with an alkylene oxide. In addition, two-ended end capping, for example, can be produced by reacting the free block copolymer with dialkyl sulfate, C₁₋₁₀-alkyl halides, C₁₋₁₀-phenyl halides, preferably chlorides, bromides, particularly preferably cyclohexyl chloride, cyclohexyl bromide, phenyl chloride or phenyl bromide.

Individual nonionic wetting agents or a combination of different nonionic surfactants can also be used. It is possible to use nonionic wetting agents from only one class, in particular only alkoxylated C₄-C₂₂-alcohols. However, as an alternative, wetting agent mixtures from various classes can also be used.

The concentration of nonionic wetting agent in the composition according to the invention can vary as a function of the leaching conditions, in particular as a function of the material to be leached.

Suitable anionic wetting agents are alkanesulfonates such as C₈-C₂₄-, preferably C₁₀-C₁₈-alkanesulfonates, and also soaps such as the Na and K salts of saturated and/or unsaturated C₈-C₂₄-carboxylic acids.

Further suitable anionic wetting agents are linear C₈-C₂₀-alkylbenzenesulfonates (“LAS”), preferably linear C₉-C₁₃-alkylbenzenesulfonates and -alkyltoluenesulfonates.

### Thickeners

Thickeners are compounds which increase the viscosity of the chemical composition. Nonlimiting examples are given, for example, in WO 2009/090169 A1: polyacrylates and hydrophobically modified polyacrylates. The advantage of the use of thickeners is that liquids having a relatively high viscosity have a higher residence time on inclined or vertical surfaces than liquid having a lower viscosity. This increases the interaction time between composition and surface.

Further particularly preferred thickeners are, for example, bentonite, xanthan and cellulose and also cellulose derivatives, in particular cellulose ethers and cellulose esters, in particular methylcellulose, hydroxyethylcellulose and carboxymethylcellulose. Further examples of thickeners are polyacrylamides, polyethers or associative polyurethane thickeners, polyvinyl alcohols and polyvinylpyrrolidones.

### Dispersants/Scale Inhibitors

Furthermore, it is possible, according to the invention, to make additional use of at least one dispersant, for example selected from the group consisting of salts of naphthalenesulfonic acids, condensation products of naphthalenesulfonic acids and formaldehyde and also polycarboxylates. Dispersants of this type are commercially available, for example, under the trade names Tamol®, Sokalan® and Nekal® from BASF SE and under the trade name Solsperse® from Lubrizol. These dispersants may also act as scale inhibitors (deposit preventers) since they disperse the calcium carbonate CaCO₃ formed in alkaline medium and thus prevent, for example, blockage of nozzles or formation of deposits in pipes. Independently of this, the composition according to the invention can additionally comprise at least one further scale inhibitor. Suitable scale inhibitors are described, for example, in WO 04/099092, which describes (meth)acrylic acid copolymers which comprise
(a) from 50 to 80% by weight, preferably from 50 to 75% by weight, particularly preferably from 55 to 70% by weight, of a poly(meth)acrylic acid skeleton,
(b) from 1 to 40% by weight, preferably from 5 to 20% by weight, particularly preferably from 7 to 15% by weight, of at least one unit which is selected from the group consisting of isobutene units, terelactone units and isopropanol units and is bound to the skeleton and/or incorporated into the skeleton and
(c) from 5 to 50% by weight, preferably from 5 to 40% by weight, particularly preferably from 10 to 30% by weight, of amide units based on aminoalkylsulfonic acids,
   where the total weight of the units in the (meth)acrylic acid copolymer is 100% by weight and all percentages by weight are based on the (meth)acrylic acid copolymer.

The (meth)acrylic acid copolymers provided according to WO 04/099092 preferably have a weight average molecular weight of the polymer comprising sulfone groups of from 1000 to 20 000 g/mol and can preferably be prepared by means of the following process steps:
(1) free-radical polymerization of (meth)acrylic acid in the presence of isopropanol and optionally water, resulting in a polymer I, and
(2) amidation of the polymer I from process step (1) by reaction with at least one aminoalkanesulfonic acid.

Further suitable scale inhibitors are, for example:
semiamides of polycarboxylic acids, which can be obtained by reaction of polymers comprising anhydride groups with compounds comprising amino groups (as described in DE 195 48 318),
vinyllactic acid and/or isopropenyllactic acid (as described in DE 197 195 16),
homopolymers of acrylic acid (as described in U.S. Pat. No. 3,756,257),
copolymers of acrylic acid and/or (meth)acrylic acid and vinyllactic acid and/or isopropenyllactic acid,
copolymers of styrene and vinyllactic acid,
copolymers of maleic acid and acrylic acid,
water-soluble or water-dispersible graft polymers, which can be obtained by free-radically initiated graft polymerization of
   (I) at least one monoethylenically unsaturated monomer,
   (II) polymers having a molar mass of from 200 to 5000 g/mol of monoethylenically unsaturated dicarboxylic acids or anhydrides thereof,
   (III) where from 5 to 20 000 parts by weight of (I) are used per 100 parts by weight of the graft base (II) (DE 195 03 546),
optionally hydrolyzed polymaleic anhydrides and salts thereof (as described in U.S. Pat. No. 3,810,834, GB-A-1 454 657 and EP-A-0 261 589),
iminodisuccinates (as described in DE 101 02 209),
formulations comprising complexing agents such as ethylenediaminetetraacetic acid (EDTA) and/or diethylenetriaminepentaacetic acid (DTPA) (as described in U.S. Pat. No. 5,366,016),
phosphonates,
polyacrylates,
polyaspartic acids or polyaspartic acids which have been modified as described in DE-A-44 34 463,
polyaspartimides,
polymers comprising hydroxamic acid, hydroxamic ether and/or hydrazide groups (as described in DE 44 27 630),
optionally hydrolyzed polymers of maleimide (as described in DE 43 42 930),
naphthylamine polycarboxylates (as described in EP 0 538 969),
oxaalkanepolyphosphonic acids (as described in EP 330 075),
polyhydroxyalkaneaminobismethylenephosphonic acids (as described in DE 40 16 753) and
oxidized polyglucosanes (as described in DE 43 30 339).

Particularly preferred dispersants are polyacrylic acid, for example the Sokalan® grades from BASF SE, and polyaspartic acids, in particular β-polyaspartic acids, having a molecular weight of from 2000 to 10 000 g/mol. Preferred polymeric compounds comprising carboxylic acid groups are the acrylic acid homopolymers indicated in EP 2 083 067 A1. These preferably have a number average molecular weight in the range from 1000 to 50 000, particularly preferably from 1500 to 20 000. Homopolymers of acrylic acid which are particularly suitable as polymeric compounds comprising carboxylic acid groups are the Sokalan® PA grades from BASF SE.

Further suitable polymeric compounds comprising carboxylic acid groups are oligomaleic acids as are described, for example, in EP-A 451 508 and EP-A 396 303.

Other compounds which are preferred as polymeric compounds comprising carboxylic acid groups are copolymers comprising at least one unsaturated monocarboxylic or dicarboxylic acid or a dicarboxylic anhydride or a salt thereof as monomer A) and at least one comonomer B) in copolymerized form. The monomer A) is preferably selected from among C₃-C₁₀-monocarboxylic acids, salts of C₃-C₁₀-monocarboxylic acids, C₄-C₈-dicarboxylic acids, anhydrides of C₄-C₈-dicarboxylic acids, salts of C₄-C₈-dicarboxylic acids and mixtures thereof. Monomers A) in salt form are preferably used in the form of their water-soluble salts, in particular the alkali metal salts such as potassium and especially sodium salts or the ammonium salts. The monomers A) can in each case be entirely or partly present in anhydride form. Of course, it is also possible to use mixtures of monomers A).

The monomers (A) are preferably selected from among acrylic acid, methacrylic acid, crotonic acid, vinylacetic acid, maleic acid, maleic anhydride, fumaric acid, citraconic acid, citraconic anhydride, itaconic acid and mixtures thereof. Particularly preferred monomers A) are acrylic acid, methacrylic acid, maleic acid, maleic anhydride and mixtures thereof. These copolymers preferably comprise at least one monomer A) in an amount of from 5 to 95% by weight, particularly preferably from 20 to 80% by weight, in particular from 30 to 70% by weight, based on the total weight of the monomers used for the polymerization, in copolymerized form.

### Corrosion Inhibitors

The agents, e.g. carboxylic acids, indicated in, for example, WO 2008/071582 A1 act as corrosion inhibitors. These can be linear or branched. Mixtures of various carboxylic acids can be particularly preferred. Caprylic acid, ethylhexanoic acid, isononanoic acid and isodecanoic acid are particularly preferred carboxylic acids. Since corrosion protection emulsions are frequently neutral to weakly alkaline, it can be advantageous to use the carboxylic acids at least partly in neutralized form, i.e. as salt. Sodium hydroxide and/or potassium hydroxide and also alkanolamines are particularly suitable for neutralization. Particular preference is given to using monoalkanolamines and/or trialkanolamines. The use of dialkanolamines is less preferred because of the risk of formation of nitrosamines. Dialkanolamines can be used equally well either alone or together with monoalkanolamines and/or trialkanolamines for neutralization.

Suitable corrosion inhibitors are, in particular:
Aliphatic carboxamides having from 14 to 36 carbon atoms, for example myristamide, palmitamide and oleamide; alkenylsuccinamides having from 6 to 36 carbon atoms, for example octenylsuccinamide, dodecenylsuccinamide; mercatobenzothiazoles.
Particularly preferred corrosion inhibitors are alkylene oxide adducts with aliphatic amines, in particular triethanolamines and ethylenediamine adducts with from 2 to 8 mol % of propylene oxide.

### Complexing Agents

Complexing agents are compounds which bind cations. Typical examples are: EDTA (N,N,N′,N′-ethylenediaminetetraacetic acid), NTA (N,N,N-nitrilotriacetic acid), MGDA (2-methylglycine-N,N-diacetic acid), GLDA (glutamic acid diacetate), ASDA (aspartic acid diacetate), IDS (iminodisuccinate), HEIDA (hydroxyethylimine diacetate), EDDS (ethylenediamine disuccinate), citric acid, oxodisuccinic acid and butanetetracarboxylic acid and completely or partially neutralized alkali metal or ammonium salts thereof.

### Other Additives

Further suitable additives are, for example, bonding agents. Suitable bonding agents are, for example, the amphiphilic water-soluble alkoxylated polyalkyleneimines of the general formula AI indicated in WO 2006/018856 A2

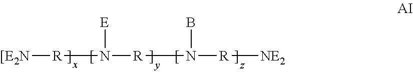

where the variables have the following meanings:
the radicals R are identical or different, linear or branched C2-C6-alkylene radicals;
B is a branch;
E is an alkyleneoxy unit of the formula

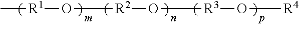

where
R1 is 1,2-propylene, 1,2-butylene and/or 1,2-isobutylene;
R2 is ethylene;
R3 is 1,2-propylene;
R4 are identical or different radicals: hydrogen; C1-C4-alkyl;
x, y, z are each from 2 to 150, where the sum x+y+z is the number of alkyleneimine units and corresponds to an average molecular weight Mw of the polyalkyleneimine before the alkoxylation of from 300 to 10 000;
m is a rational number from 0 to 2;
n is a rational number from 6 to 18;
p is a rational number from 3 to 12, where 0.8≦n/p≦1.0(x+y+z)½.

The invention further provides a slurry composed of the carrier fluid, in particular cutting fluid, abrasives, in particular grinding and/or cutting grains, and optionally additives.

It is possible to use the customary abrasives, in particular grinding and/or cutting grains, for example metal, metal or semimetal, carbide, nitride, oxide, boride or diamond grains. Particularly preferred cutting grains are carbide and boride grains, in particular silicon carbide (SiC) grains. The cutting grains preferably have a geometry matched to the materials and the wafers to be cut. A preferred particle size is in the range from 0.5 to 50 μm. The cutting grains can be present in heterodisperse or homodisperse form. The cutting grains are preferably comprised in the cutting fluid composition in a concentration of from 25 to 60% by weight, in particular from 40 to 50% by weight.

In a particularly preferred embodiment, the carrier fluid, in particular cutting fluid, has a contact angle to V2A steel of from 5 to 40°, in particular from 10 to 30°. The contact angle is determined here at 25° C. on a steel plate made of V2A steel whose surface has been rinsed with water and acetone.

In a further preferred embodiment, the carrier fluids, in particular cutting fluids, of the invention lead to an average weight decrease over two tests of not more than 20-60 mg in one minute on a stainless steel cylinder M1M6/05R, Torrington, having a diameter of 12 mm on a MDD2 balance from Hermann Reichert Maschinenbau, Heidenhof Backnang, at a loading of 300 N and over a distance of 110 m.

In a further preferred embodiment, the carrier fluids, in particular cutting fluids, of the invention take up not more than 30%, preferably not more than 15%, of water after storage for 10 hours in a Heraeus BBD 6220 CO₂ incubator at 38° C. and 78% relative atmospheric humidity. For the storage test, 1 g of carrier fluid, in particular cutting fluid, in Petri dishes having an internal diameter of 60 mm is used in each case. The average of a duplicate determination is determined in each case. In a very particularly preferred embodiment, this water uptake does not increase even on further exposure.

A slurry composed of a carrier fluid, in particular cutting fluid, according to the invention and 40% by weight of the abrasives indicated below, in particular grinding and/or cutting grains, preferably has a viscosity measured at 30° C. using a Brookfield LVDV-III Ultra apparatus (spindle V-73) of from 140 to 200 mPas, in particular from 150 to 190 mPas, when Carborex BWF 800 PV silicon carbide grains from Washington Mills are used.

The invention further relates a method of cutting wafers of, in particular, inorganic semiconductors such as silicon ingots or silicon blocks by means of a wire saw using a slurry based on the cutting fluid of the invention and cutting grains.

The invention further relates to a method of grinding or polishing materials derived from, for example, silicon ingots or blocks by, for example, chemomechanical polishing (CMP) or of grinding polymers, in particular for lenses, using abrasives dispersed in a carrier fluid to be used according to the invention.

### Advantages

The carrier fluid, in particular cutting fluid, of the invention and the cutting method of the invention are particularly suitable for sawing ingots, blocks or cylinders of monocrystalline or polycrystalline silicon single crystals or polycrystals, GaAs, CdTe and other semiconductors and ceramics.

The carrier fluid, in particular cutting fluid, of the invention displays little or no foaming, does not require any additives, is pH neutral and is nontoxic. In addition, it does not contain any volatile organic constituents. Furthermore, the carrier fluid, in particular cutting fluid, of the invention is highly suitable for reprocessing by means of a wet chemical work-up, for example as described in WO 02/40407 A1 and EP 1 390 184 A1.

### EXAMPLES

### General Method of Preparing Polyethers

1-2 mol of the starter alcohol were in each case placed in a water-free, dry 1 l pressure reactor, admixed with 0.2% by weight (based on end product) of KOH and flushed with nitrogen. The closed reactor was then heated to 130° C. over a period of 30 minutes and a gauge pressure of 1 bar was set by means of nitrogen. The molar amounts of propylene oxide (hereinafter PO) and ethylene oxide (hereinafter EO) indicated in table 1 were subsequently metered in in parallel (random process) or in succession (block process) while stirring. In the block process, after PO had been added and a constant pressure had been reached, the mixture was stirred at 130° C. for at least ½ hour and the pressure was set to 1 bar before the addition of EO. The vessel was thermostated to 130° C. during the reaction. After a constant pressure had been reached, the mixture was stirred for a further about ½ hour. After the reaction was complete, the mix was cooled to 80° C., the reactor was depressurized and flushed with nitrogen, the amount of glacial acetic acid calculated for neutralization of the KOH was added and the mixture was stirred for ½ hour.

The OH number was determined in accordance with DIN 51562, the residual alcohol content was determined by means of gas chromatography and the APHA color number was determined in accordance with EN 1557 (at 23° C.).

TABLE 1
Examples and analytical characterization

| | Chemical composition | | | |
|---|---|---|---|---|
| | (PO here is propyleneoxy, | OH | Residual | Color |
| Product | EO here is ethyleneoxy) | number | alcohol % | number |
| Pluriol ® | polyethylene glycol 200 | 560 | DEG3 | max 30 |
| E 200 | | | | |
| C1 | (3-methylbutan-1-ol) + | 98.3 | 0.2 | 70 |
| | 1.6 PO + 8.3 EO | | | |
| | block process | | | |
| C2 | (3-methylbutan-1-ol) + | 87.0 | <0.1 | 25 |
| | 5.5 PO + 5.5 EO | | | |
| | random process | | | |
| C3 | (3-Methylbutan-1-ol) + | 85.7 | <0.1 | 20 |
| | 6.0 PO + 5.0 EO | | | |
| | random process | | | |
| C4 | n-butan-1-ol + 5.5 PO + | 90.4 | <0.1 | 30 |
| | 5.5 EO | | | |
| | random process | | | |
| C5 | methyl diglycol + 8.8 EO/ | 106.6/ | 0.2/<0.1 | 60/50 |
| | (3-methylbutan-1-ol) + | 112.4 | | |
| | 1.5 PO + 7.0 EO | | | |
| | in a weight ratio of 6/4 | | | |
| | block process | | | |
| C6 | Pluronic ® PE 6200/ | 112.4 | 0.1 | 30 |
| | n-pentanol + | | | |
| | 1.5 PO + 6 EO | | | |
| | in a weight ratio of 18/82 | | | |
| | block process | | | |
| C7 | Plurafac ® LF 401/ | 126.2 | 0.1 | 25 |
| | n-pentanol + 1.5 PO + 6 EO | | | |
| | in a weight ratio of 3/7 | | | |
| | block process | | | |
| C8 | n-hexanol + 5.5 PO + 5.5 EO | 84.0 | <0.1 | 20 |
| | random process | | | |
| II.1 | 2-methylbutan-1-ol + | 85.2 | <0.1 | 20 |
| | 5.5 PO + 5.5 EO | | | |
| | random process | | | |
| II.2 | 2-methylbutan-1-ol + | 86.1 | <0.1 | 15 |
| | 6.0 PO + 5.0 EO | | | |
| | random process | | | |

The wetting agents and alkylene glycols Pluronic® PE 6200 and Plurafac® LF 401 added in examples C6 and C7 are commercial products of BASF SE, Ludwigshafen. The analytical data reported are based on the component pentanol+1.5 PO+6 EO according to the invention, block process.

### Properties/Determination of the Characteristic Values

The properties of the cutting fluids according to the invention are summarized in table 2. The following properties were determined:
Water uptake

The water uptake of the cutting fluids was determined after storage in a Heraeus BBD 6220 CO₂ incubator at 38° C. and 78% relative atmospheric humidity for a time of 10 hours and 24 hours. For storage, 1 g in each case of cutting fluid was used in Petri dishes having an internal diameter of 60 mm. The average of a duplicate determination was determined in each case. The water uptake is in each case reported in percent by weight increase based on the initial weight.
Slurry viscosity

To determine the slurry viscosity, a mixture of 60 percent by weight of the sawing fluid and 40 percent by weight of SiC of the type Carborex BW F 800 PV from Washington Mills was produced and the viscosity was determined at 30° C. and optionally 50° C. using a model LVDV-III Ultra viscosimeter from Brookfield (Spindle V-73). The slurry viscosity is reported in mPas.
Contact angle

The contact angle of the cutting fluids was determined at 25° C. one second after application of a droplet to a steel plate made of V2A steel whose surface had been rinsed with water and acetone and subsequently dried in air for 1 hour. A video-aided high-speed contact angle measuring instrument from Dataphysics Instruments GmbH, Raiffeisenstraβe 34, Filderstadt was used for the determination. The unit of the contact angle is °.
Abrasion

The abrasion behavior was determined on a frictional wear balance MDD2 from Hermann Reichert Maschinenbau, Heidenhof Backnang, at a loading of 300 N and a test distance of 100 m in 54.5 sec. on a stainless steel cylinder M1M6/05R, Torrington having a diameter of 12 mm. A duplicate determination was carried out in each case and the average of the weight decrease of the cylinder was determined. The weight decrease is reported in mg.

TABLE 2

| | H₂O | | | |
|---|---|---|---|---|
| | uptake 10 h | Viscosity [mPas] | Contact angle | Abrasion |
| Product | (24 h) [%] | 30° C. (50° C.) | [°] | [mg] |
| Pluriol ® | | 203 (94) | 41 | 70 |
| E 200 | | | | |
| C1 | 17.2 (18.2) | 181 (85) | 30 | 25 |
| C2 | 5.7 (5.9) | 160 (89) | 23 | 32 |
| C3 | 5.3 (5.3) | 185 (161) | 21 | 22 |
| C4 | 6.6 (6.6) | 155 (93) | 23 | 28 |
| C5 | 16.3 (24.3) | 164 (77) | 29 | 35 |
| C6 | 5.5 (8.3) | 175 (84) | 28 | 29 |
| C7 | 6.3 (8.2) | 167 (84) | 36 | 33 |
| C8 | 5.3 (5.3) | 198 (176) | 34 | 38 |

Pluriol® E 200 is a polyethylene glycol having an average molar mass of 200 from BASF SE, Ludwigshafen. The example represents the prior art and is not according to the invention. The compounds 11.1 and 11.2 gave results comparable to the compounds C2 and C3.

### Practical Test

Sawing tests on polycrystalline silicon blocks were carried out on a DS 265 wire saw from Meyer Burger AG, Allmendstrasse 86, CH 3600 Thun using the sawing fluids C1 and C3. The test conditions were:

Dimensions of the wafers: 5″×5″, 150 μm
SiC grade: F 88, ds50=6.5 μm
Advance rate: 0.6 mm/s
Wire speed: 14 m/s
Wire diameter: 120 μm
Wire tension: 20 N
Slurry temperature: 22° C.
Composition of the slurry: 60% by weight of sawing fluid, 40% by weight of SiC

Compared to the PEG 200 (Pluriol® E 200) which is usually used in industrial practice, the following improvements were found for the cutting fluids according to the invention:

| | Pluriol ® | | |
|---|---|---|---|
| | E 200 | C1 | C3 |
| Water uptake, [% by weight] | 3 | <0.5 | <0.5 |
| Force per wire in advance direction, [N] | 0.62 | 0.57 | 0.58 |
| Force per wire in wire direction, [N] | 1.62 | 1.51 | 1.55 |
| Power uptake per wire, [W] | 29.5 | 24.0 | 26.5 |
| (without machine contribution) | | | |
| TTV, 5-point support, [%] | 12.5 | 11.8 | 10.5 |
| (total thickness variation of wafers) | | | |
| Critical fracture stress, [N/mm²] | 158 | 163 | 178 |

Similar improvements were also found when using the other products according to the invention shown in table 1.

1. The method of using compounds of the formula I
R¹[O(EO)ₓ(AO)_{y}H]_{z}
where
R¹ is a z-valent alkyl radical having from 1 to 20 carbon atoms
(EO) is an ethyleneoxy radical
(AO) is an alkyleneoxy radical having from 3 to 10 carbon atoms
x is an integer from 3 to 12, in particular from 5 to 10
y is an integer from 0 to 10, in particular from 4 to 8
z is an integer from 1 to 6, in particular from 1 to 3,
for producing carrier fluids for abrasives, in particular cutting fluids, having a reduced water uptake for removal of material, in particular for sawing wafers by means of a wire saw.

2. The method according to claim 1, wherein, in the formula I,
R¹ is a z-valent alkyl radical having from 5 to 10 carbons, in particular pentyl.

3. The method according to claim 1, wherein the contact angle of the cutting fluid on V2A steel at 25° C. is from 25 to 50°.

4. The method according to claim 1, wherein the wafer is a semiconductor, in particular silicon.

5. The method according to claim 1, wherein the carrier fluid, in particular cutting fluid, is used in a slurry together with cutting grains, where metal, carbide, nitride, metal oxide, boride or diamond grains are used as cutting grains.

6. The method according to claim 1, wherein the carrier fluid, in particular cutting fluid, is worked up to separate off the resulting abraded material during or after removal of the material, in particular after cutting.

7. A carrier fluid, in particular cutting fluid, comprising at least one compound of the formula I
R¹[O(EO)ₓ(AO)_{y}H]_{z},
where
R¹ is a z-valent alkyl radical having from 5 to 10 carbon atoms
(EO) is an ethyleneoxy radical
(AO) is an alkyleneoxy radical having from 3 to 10 carbon atoms
x is an integer from 3 to 12, in particular from 5 to 10
y is an integer from 0.5 to 10, in particular from 4 to 8
z is an integer from 1 to 6, in particular from 1 to 3.

8. The carrier fluid, in particular cutting fluid, according to claim 7, wherein R¹ is pentyl.

9. The carrier fluid, in particular cutting fluid, according to claim 7, wherein the contact angle of the carrier fluid, in particular cutting fluid, on V2A steel at 25° C. is from 25 to 50°.

10. The method of using a carrier fluid, in particular cutting fluid, according to claim 7 for the removal of material, in particular during sawing of wafers, from an object to be cut, in particular semiconductor, in particular silicon, by means of a saw using cutting grains, in particular silicon carbide grains.

11. The method of using a carrier fluid according to claim 7 for polishing materials, in particular for polishing wafers of silicon or of materials composed of polymers, in particular for lens production.

12. A method of cutting wafers from an object by means of a saw using a slurry composed of a cutting fluid and cutting grains, in particular cutting grains composed of metal, a carbide, nitride, oxide, boride, α-alumina or diamond, wherein the cutting fluid is a cutting fluid according to claim 7 which is optionally worked up to separate off the resulting abraded material during or after removal of the material, in particular after cutting.

13. A method of polishing materials, in particular materials composed of silicon or polymers, using a slurry composed of a carrier fluid and abrasive materials, wherein a carrier fluid according to claim 7 is used as carrier fluid.

14. A wafer, in particular silicon wafer, obtained by the method according to claim 12.

15. A compound of the formula II
R¹O(EO)ₓ(AO)_{y}H
where
R¹ is 2-methylbutyl or 3-methylbutyl and
(EO), (AO), x and y are as defined in claim 1.
